(19) United States
(12) Patent Application Publication
(10) Pub. No.: US 2025/0299514 A1
(43) Pub. Date: Sep. 25, 2025

(54) PERSON IDENTIFICATION METHOD BASED ON GAIT ANALYSIS

(71) Applicant: National Tsing Hua University, Hsinchu City (TW)

(72) Inventors: Yun-Ju LEE, Hsinchu City (TW); Yu-Sen WU, Hsinchu City (TW)

(73) Assignee: National Tsing Hua University, Hsinchu City (TW)

(21) Appl. No.: 18/673,080

(22) Filed: May 23, 2024

(30) Foreign Application Priority Data
Mar. 22, 2024 (TW) 113110698

(51) Int. Cl. G06V 40/20; A61B 5/11; A61B 5/117; G06V 10/32

(52) U.S. Cl. CPC G06V 40/25; A61B 5/112; A61B 5/117; G06V 10/32

(57) ABSTRACT

A person identification method for determining an identity of a person is implemented by a processor. The method includes: obtaining a gait dataset that is related to the person; obtaining a group determination based on a first gait recognition model, a second gait recognition model and the gait dataset, where the group determination indicates whether the person belongs to a group that includes a plurality of predetermined members; and when the group determination indicates that the person belongs to the group, obtaining an identity determination based on a third gait recognition model and the gait dataset, where the identity determination indicates which one of the predetermined members the person is.

### CROSS-REFERENCE TO RELATED APPLICATION

This application claims priority to Taiwanese Invention Patent Application No. 113110698, filed on Mar. 22, 2024, the entire disclosure of which is incorporated by reference herein.

### FIELD

The disclosure relates to identity recognition, and more particularly to a person identification method for determining an identity of a person based on gait analysis.

### BACKGROUND

Application of gait analysis technology in biometric authentication has become a trend in recent years. Compared to a conventional biometric authentication method, which collects features of a user such as fingerprint, face, iris, etc., using an authentication device that is based on gait analysis technology has the following advantages: (1) gait data is a dynamic biometric feature that is more likely to be recognized by the authentication device even with obstruction, (2) gait data cannot be easily copied or stolen through images, and/or (3) users are not required to make physical contact with the authentication device for authentication.

### SUMMARY

Therefore, an object of the disclosure is to provide a person identification method for determining an identity of a person based on gait analysis that has a higher accuracy compared to the prior art.

According to the disclosure, the person identification method is implemented by a processor and includes: obtaining a gait dataset that is related to the person; obtaining a group determination based on a first gait recognition model, a second gait recognition model and the gait dataset, where the group determination indicates whether the person belongs to a group that includes a plurality of predetermined members; and when the group determination indicates that the person belongs to the group, obtaining an identity determination based on a third gait recognition model and the gait dataset, where the identity determination indicates which one of the predetermined members the person is.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating an identity recognition system implementing a person identification method for determining an identity of a person according to an embodiment of the disclosure.

FIG. 2 is a flow chart illustrating the person identification method according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

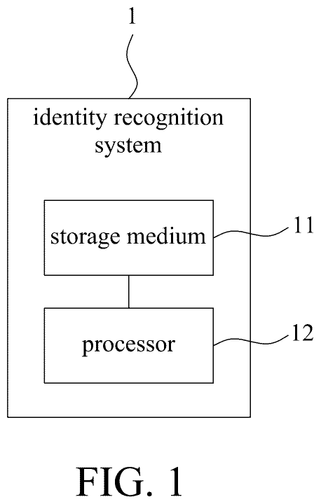

Referring to FIG. 1, according to an embodiment of the disclosure, an identity recognition system 1 implementing a person identification method for determining an identity of a person based on gait analysis is provided. The identity recognition system 1 may be implemented as a door lock system that is applied to a household for controlling entry of family members, or that is applied to an institution for controlling entry of staff members. The identity recognition system 1 includes a storage medium 11, and a processor 12 that is electrically connected to the storage medium 11. The storage medium 11 may be embodied using computer-readable storage mediums such as hard disk drive(s), random access memory (RAM), read only memory (ROM), programmable ROM (PROM), and/or flash memory, etc. The processor 12 may be, but is not limited to, a single core processor, a multi-core processor, a dual-core mobile processor, a microprocessor, a microcontroller, a digital signal processor (DSP), a field-programmable gate array (FPGA), an application specific integrated circuit (ASIC), a radio-frequency integrated circuit (RFIC), and/or a system on a chip (SoC), etc.

The storage medium 11 stores a plurality of training datasets of gaits corresponding respectively to a plurality of predetermined members. Each of the training datasets of gaits includes a vector that indicates a plurality of gait features of the corresponding one of the predetermined members, and the vector includes a plurality of training values corresponding respectively to the gait features.

In this embodiment, the gait features may be related to accelerations on X, Y and Z axes and angular velocities about the X, Y and Z axes. To describe in further detail, when a user steps on a sensor mat, the gait features may be obtained by a three-axis accelerometer of the sensor mat as an X-axis acceleration signal, a Y-axis acceleration signal, and a Z-axis acceleration signal, and may further be obtained by a three-axis gyroscope of the sensor mat as an X-axis angular velocity signal, a Y-axis angular velocity signal, and a Z-axis angular velocity signal.

The predetermined members are set to be in a group. In one example, when the identity recognition system 1 is applied to a company, the group may include all staff members of the company (i.e., the staff members corresponding respectively to the predetermined members).

The storage medium 11 further stores a first gait recognition model, a second gait recognition model, and a third gait recognition model. In this embodiment, the first gait recognition model was trained, based on the training datasets of gaits, using a convolutional neural network (CNN) algorithm and a long short-term memory (LSTM) algorithm; the second gait recognition model was trained, based on the training datasets of gaits, using a gated recurrent unit (GRU) algorithm; and the third gait recognition model is identical to the first gait recognition model. The CNN algorithm is, for example, a 1-dimensional CNN.

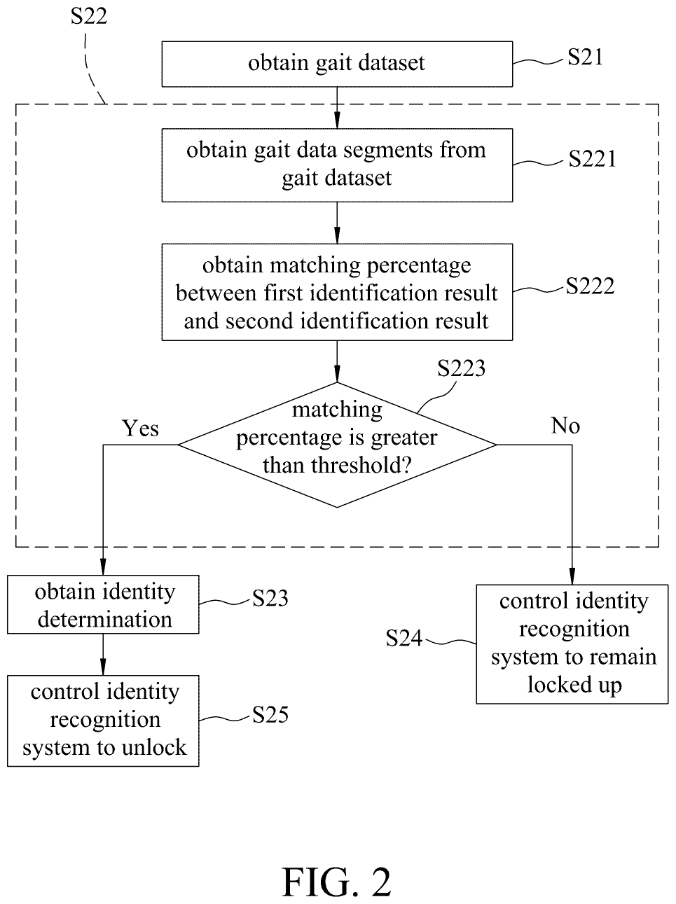

Referring further to FIG. 2, the person identification method for determining an identity of a person based on gait analysis is performed by the processor 12, and a flow of the method includes steps S21 to S25.

In step S21, the processor 12 obtains a gait dataset that is related to the person during a period of time (referred to as “stepping period” hereinafter). In one example, the gait dataset is obtained by the three-axis accelerometer and the three-axis gyroscope of the sensor mat when the user takes a step (e.g., taking 1.5 seconds, which is the stepping period in this example) on the sensor mat, and a signal transmitter of the sensor mat transmits the gait dataset to the processor 12 through wireless technologies, such as but not limited to, Wi-Fi, Bluetooth, cellular networks, etc.

In this embodiment, the gait dataset includes a plurality of feature values related to the gait features of the user during the stepping period. Specifically, the gait dataset includes a plurality of pieces of data corresponding respectively to a plurality of time points that are within the stepping period. Each of the pieces of data includes those of the feature values corresponding respectively to the gait features at the corresponding one of the time points.

In step S22, the processor 12 obtains a group determination based on the first gait recognition model, the second gait recognition model and the gait dataset. The group determination indicates whether the person belongs to the group that includes the predetermined members. Specifically, step S22 includes sub-steps S221 to S223.

In sub-step S221, the processor 12 obtains a plurality of gait data segments that are consecutive in time from the gait dataset, where each of the gait data segments includes a plurality of segment values corresponding respectively to the gait features.

In one example, the processor 12 obtains the gait data segments by processing the gait dataset using a sliding window technique. To describe in further detail, assuming that the stepping period includes N time points, where N is a positive integer that is greater than 50, and that a window to be used is predetermined to have a window size of 50 time points, the gait data segments are obtained by moving the window over the pieces of data of the gait dataset with respect to time, and an n^{th} one of the gait data segments obtained by the window corresponds to the time points [n, n+50−1] (i.e., from the n^{th} time point to the (n+50-1)^{th} time point of the stepping period), where n is a positive integer from 1 to (N−50+1). As a result, the segment values of each of the gait data segments segmented by the window correspond to those of the time points from an n^{th} one of the time points to an (n+50−1)^{th} one of the time points. Specifically, the segment values are related to those of the pieces of data that correspond to those of the time points the gait data segment corresponds to (i.e., those of the time points from an n^{th} one of the time points to an (n+50−1)^{th} one of the time points). For each of the gait features of each of the gait data segments, the processor 12 obtains the segment value that corresponds to the gait feature by, for example, calculating an average of those of the feature values that correspond to the gait feature at those of the time points of the gait data segment.

In sub-step S222, the processor 12 obtains a first identification result based on the gait data segments and the first gait recognition model, and obtains a second identification result based on the gait data segments and the second gait recognition model. The processor 12 further obtains a matching percentage between the first identification result and the second identification result.

To describe in further detail, when obtaining the first identification result, for each of the gait data segments, the processor 12 first performs feature scaling on the segment values of the gait data segment, so as to obtain a plurality of first scaling values corresponding respectively to the segment values, the processor 12 then obtains a first identity based on the first scaling values and the first gait recognition model. When obtaining the second identification result, for each of the gait data segments, the processor 12 first performs feature scaling on the segment values of the gait data segment, so as to obtain a plurality of second scaling values corresponding respectively to the segment values, the processor 12 then obtains a second identity based on the second scaling values and the second gait recognition model. Specifically, the first identity indicates which one of the predetermined members the person is based on the first gait recognition model, and the second identity indicates which one of the predetermined members the person is based on the second gait recognition model. The first identities obtained for the gait data segments cooperatively form the first identification result, and the second identities obtained for the gait data segments cooperatively form the second identification result.

When obtaining the matching percentage between the first identification result and the second identification result, for each of the gait data segments, the processor 12 obtains either a positive determination or a negative determination based on the first identity and the second identity. Specifically, the positive determination is obtained when both the first identity and the second identity indicate a same one of the predetermined members, and the negative determination is obtained when the first identity and the second identity indicate different ones of the predetermined members. The processor 12 then obtains the matching percentage based on the positive determination or the negative determination obtained for each of the gait data segments.

In one example, assuming that the predetermined members in the group include person A, person B, person C, and person D. For a first one of the gait data segments (segment one), if the first identity obtained for segment one is “person A”, and the second identity obtained for segment one is “person C”, then the processor 12 obtains the negative determination (e.g., labelled as “0”) for segment one. For a second one of the gait data segments (segment two), if the first identity obtained for segment two is “person A”, and the second identity obtained for segment two is also “person A”, then the processor 12 obtains the positive determination (e.g., labelled as “1”) for segment two. After obtaining the first identity and the second identity for each of the gait data segments, the processor 12 may calculate the matching percentage as a number of positive determinations (i.e., a number of those of the gait data segments that are labelled as “1”) divided by a sum of the number of positive determinations and a number of negative determinations (i.e., a number of those of the gait data segments that are labelled as “0”).

In this embodiment, the first scaling values are obtained using binary transform feature scaling, which scales those of the segment values that are greater than zero to be “+1”, and scales those of the segment values that are smaller than zero to be “−1”. In this embodiment, the second scaling values are obtained using min-max normalization feature scaling, which scales a largest one of the segment values to be “+1”, scales a smallest one of the segment values to be “−1”, and scales the rest of the segment values between “−1” and “+1”.

In sub-step S223, the processor 12 obtains the group determination based on the matching percentage. In this embodiment, the processor 12 obtains the group determination by determining whether the matching percentage is greater than a threshold (e.g., 0.85). When the matching percentage is greater than the threshold, the group determination indicates that the person belongs to the group, and the flow proceeds to step S23. Otherwise, the group determination indicates that the person does not belong to the group, and the flow proceeds to step S24.

In step S23, the processor 12 obtains an identity determination based on the third gait recognition model and the gait dataset, where the identity determination indicates which one of the predetermined members the person is.

In one example, the processor 12 obtains the gait data segments from the gait dataset as described earlier in sub-step S221, and for each of the gait data segments, the processor 12 performs feature scaling on the segment values of the gait data segments, so as to obtain a plurality of third scaling values corresponding respectively to the segment values. In some examples, the gait data segments that were obtained in sub-step S221 may be directly used in step S23. The processor 12 then obtains a third identity based on the third scaling values and the third gait recognition model. Finally, the processor obtains the identity determination based on the third identities obtained for the gait data segments. The flow then proceeds to step S25.

In this embodiment, since the third gait recognition model is the same as the first gait recognition model, the third scaling values are also obtained using the binary transform feature scaling. In some examples, the first scaling values may be directly used as the third scaling values. In some examples, the first identities obtained in sub-step S222 may be directly used as the third identities. Furthermore, the identity determination indicates one of the predetermined members that appeared the most often among the third identities obtained for the gait data segments.

In step S24, the processor 12 executes an action in response to determining that the person does not belong to the group. For example, the processor 12 may control the identity recognition system 1 to remain locked up when the identity recognition system 1 is implemented as the door lock system.

In step S25, the processor 12 executes another action in response to determining that the person belongs to the group, and that the identity determination has been obtained. For example, the processor 12 may control the identity recognition system 1 to unlock when the identity recognition system 1 is implemented as the door lock system.

In some embodiments, the processor 12 may control the identity recognition system 1 to unlock after step S223 and before step S23, and after step S23, the processor 12 may control the identity recognition system 1 to record clock in/clock out information of the person based on the identity determination when the identity recognition system 1 is applied to an institution, or to perform personalized setting for the person based on the identity determination when the identity recognition system 1 is applied to a household.

In summary, according to the disclosure, in a first stage, the processor 12 determines whether the person belongs to the group or not based on the first gait recognition model and the second gait recognition model, and in a second stage, the processor 12 determines which one of the predetermined members the person is based on the third gait recognition model. By performing the method in the abovementioned two stages, accuracy of identifying the person may be increased compared to when only performing the second stage.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A person identification method for determining an identity of a person, implemented by a processor and comprising:
obtaining a gait dataset that is related to the person;
obtaining a group determination based on a first gait recognition model, a second gait recognition model and the gait dataset, where the group determination indicates whether the person belongs to a group that includes a plurality of predetermined members; and
when the group determination indicates that the person belongs to the group, obtaining an identity determination based on a third gait recognition model and the gait dataset, where the identity determination indicates which one of the predetermined members the person is.

2. The method as claimed in claim 1, wherein the obtaining of the group determination includes:
obtaining a plurality of gait data segments from the gait dataset, where each of the gait data segments includes a plurality of segment values corresponding respectively to a plurality of gait features;
obtaining a first identification result based on the gait data segments and the first gait recognition model;
obtaining a second identification result based on the gait data segments and the second gait recognition model;
obtaining a matching percentage between the first identification result and the second identification result; and
obtaining the group determination based on the matching percentage.

3. The method as claimed in claim 2, wherein:
the obtaining of the first identification result includes, for each of the gait data segments, obtaining a first identity based on the segment values that are included in the gait data segment and the first gait recognition model, where the first identity indicates which one of the predetermined members the person is;
the obtaining of the second identification result includes, for each of the gait data segments, obtaining a second identity based on the segment values that are included in the gait data segment and the second gait recognition model, where the second identity indicates which one of the predetermined members the person is;
the obtaining of the matching percentage includes,
for each of the gait data segments, obtaining one of a positive determination and a negative determination based on the first identity and the second identity, where the positive determination is obtained when both the first identity and the second identity indicate a same one of the predetermined members, and the negative determination is obtained when the first identity and the second identity indicate different ones of the predetermined members, and
obtaining the matching percentage based on the one of the positive determination and the negative determination obtained for each of the gait data segments; and
the first identities obtained for the gait data segments cooperatively form the first identification result, and the second identities obtained for the gait data segments cooperatively form the second identification result.

4. The method as claimed in claim 3, wherein the obtaining of the first identity includes, for each of the gait data segments:
performing feature scaling on the segment values of the gait data segment, so as to obtain a plurality of scaling values corresponding respectively to the segment values; and
obtaining the first identity based on the scaling values and the first gait recognition model.

5. The method as claimed in claim 3, wherein the obtaining of the second identity includes, for each of the gait data segments:
performing feature scaling on the segment values of the gait data segment, so as to obtain a plurality of scaling values corresponding respectively to the segment values; and
obtaining the second identity based on the scaling values and the second gait recognition model.

6. The method as claimed in claim 1, wherein the obtaining of the identity determination includes:
obtaining a plurality of gait data segments from the gait dataset, where each of the gait data segments includes a plurality of segment values corresponding respectively to a plurality of gait features;
for each of the gait data segments, obtaining an identity based on the segment values that are included in the gait data segment and the third gait recognition model, where the identity indicates which one of the predetermined members the person is; and
obtaining the identity determination based on the identities obtained for the gait data segments.

7. The method as claimed in claim 6, wherein the obtaining of the identity includes, for each of the gait data segments:
performing feature scaling on the segment values of the gait data segment, so as to obtain a plurality of scaling values corresponding respectively to the segment values; and
obtaining the identity based on the scaling values and the third gait recognition model.

8. The method as claimed in claim 1, wherein each of the first gait recognition model, the second gait recognition model, and the third gait recognition model was trained based on a plurality of training datasets of gaits corresponding respectively to the predetermined members.
